(19) United States
(12) Patent Application Publication
(10) Pub. No.: US 2006/0228714 A1
(43) Pub. Date: Oct. 12, 2006

(54) Nucleic acid representations utilizing type IIB restriction endonuclease cleavage products

(75) Inventors: Matthew L. Meyerson, Concord, MA (US); Torstein Tengs, Allston, MA (US)

Correspondence Address: PALMER & DODGE, LLP;KATHLEEN M. WILLIAMS, 111 HUNTINGTON AVENUE, BOSTON, MA, 02199, US

(73) Assignee: Dana Farber Cancer Institute

(21) Appl. No.: 11/058,729

(22) Filed: Feb. 15, 2005

Related U.S. Application Data
(60) Provisional application No. 60/545,047, filed on Feb. 17, 2004.

(51) Int. Cl. C40B 40/08; C12P 19/34

(52) U.S. Cl. 435/6; 435/91.2

(57) ABSTRACT

This invention encompasses nucleic acid libraries comprising Type IIB restriction endonuclease cleavage products, or tags, including concatenated tags, and using concatenated and single Type IIB restriction endonuclease cleavage tags in diverse methods including karyotyping, pathogen discovery, identification of novel genes, subtraction techniques and transcript profiling.

### RELATED APPLICATIONS

This application is a utility patent application which claims the benefit of U.S. Provisional Application No. 60/545,047, filed on Feb. 17, 2004. This application is incorporated herein by reference in it's entirety.

### GOVERNMENT SUPPORT

The invention was supported in part by a grant R01 CA098185 from the National Cancer Institute. The Government has certain rights in the invention.

### BACKGROUND

With the sequencing of the human genome (Lander, 2001; Venter, 2001), it is now possible to apply high-throughput genomics methods to the understanding, analysis, and diagnosis of human diseases. These applications include the discovery, detection, and diagnosis of novel microbial pathogens, of human genetic variation, and of cancer-specific alterations in genome structure and in gene transcription.

Many of these high-throughput molecular genomic approaches have been developed relying on the idea of representational subsampling to screen a large number of nucleic acid loci, either through sequencing or hybridization. The concept behind subsampling is that it is possible to analyze a large, complex genome or set of transcripts by detailed analyses of a representational subset of the starting material. Serial analysis of gene expression, or SAGE (Velculescu et al. 1995), relies on analyses of concatenates of short cDNA tags to do transcriptional profiling, whereas Digital Karyotyping (Wang at al. 2003) uses the same technique to karyotype genomes and look for loci that are amplified or (partially) deleted. Various array-based approaches have also been developed to analyze transcriptomes and genomes. These methods rely on hybridization of nucleic acids to probes of a genomic representation that are deposited on arrays. Hybridization between probe and template can be detected specifically and thus show presence or absence of nucleic acids complementary to the probes. These different approaches use specific strategies to obtain the representational material so that it can be hybridized or concatenated and sequenced.

With the availability of the human genome sequence, sequencing only a short stretch of specific nucleic acids should be sufficient in theory to identify all genes expressed in a biological sample. The Expressed Sequence Tag (EST) generates relatively short cDNA fragments from 3′ ends of transcripts that can be used for identifying a full-length gene. However, the EST method still utilizes one cDNA per clone, which means one sequencing reaction yields one cDNA sequence. An effective way to improve this yield so that each plasmid and each sequencing reaction yields many cDNA sequences is to link together short cDNA fragments from end to end. The Serial Analysis Gene Expression (SAGE) method effectively utilizes such a concatenation procedure. SAGE-related U.S. Pat. Nos. 5,695,937; 5,866,330; 6,383,743; 6,461,814; 5,968,784 and 6,498,013.

### SUMMARY OF THE INVENTION

This invention encompasses nucleic acid libraries comprising Type IIB restriction endonuclease cleavage products, or tags, including concatenated tags, and using concatenated and single tags in methods such as karyotyping, pathogen discovery, identification of novel genes, subtraction techniques and transcript profiling.

Type IIB Restriction Enzyme Tags

Type IIB restriction endonuclease digestion products serve as the foundation of the instant invention.

“Type IIB restriction endonucleases” are defined as site-specific endonucleases that cut both strands of double-stranded DNA upstream and downstream of their recognition sequences (Roberts et al. 2003, (Nucleic Acids Research, 2003, 31(7):1805-1812), FIG. 1). Type IIB restriction enzymes produce DNA fragments which are of uniform length, greater than 20 base pairs in length, and which are generated from throughout the entire length of a genomic DNA or cDNA. In a preferred embodiment, the type IIB restriction enzyme used to generate the tags is selected from the group consisting of AloI, PpiI, PsrI, BaeI, BplI, FalI, BcgI, Bsp24I, BsaXI, CjeI, CjePI, HaeIV and Hin4I. All described Type IIB enzymes leave a 3′ overhang after cutting, and released tags range in size from 32 to 27 bases (without cohesive ends), depending on which enzyme is used. Recognition sequences are interrupted and range from about 5 to 7 nucleotides. Hitherto-undiscovered type IIB enzymes may have different properties. Some recognition sequences are symmetrical, whereas others are not. Non-symmetrical cutters will have approximately twice the cutting frequency of an enzyme that recognizes palindromic sequences. Some Type IIB enzymes also exhibit star-activity. The enzymes listed in FIG. 2 will thus have a theoretical cutting frequency ranging from one site per 4⁷/2=8192 bases to one site per 341+1/3 bases, although other yet undescribed enzymes may have different cutting frequencies. Type IIB restriction enzymes are available commercially, through companies such as Fermentas, SibEnzyme and New England Biolabs.

Digestion of DNA with Type IIB restriction enzymes generate DNA fragments that have the property of containing nucleotides of unspecified sequence by virtue of the enzymatic cutting outside of the recognition site of the enzyme. These fragments produced by digestion with Type IIB restriction enzymes generate fragments long enough so that the unspecified sequences can be confidently identified with the full length sequence of the genomic or cDNA molecule from which each was derived.

A “type IIB restriction enzyme tag” or “tag” is defined as piece of DNA that has been generated by digestion of a DNA with a Type IIB restriction enzyme. Because a type IIB restriction enzyme tag cuts the DNA both upstream and downstream of its recognition sequence, a “type IIB restriction enzyme tag” or “tag” contains a Type IIB restriction enzyme recognition sequence, as well as unspecified sequence which uniquely corresponds to a segment of the DNA or cDNA subjected to digestion by the enzyme. Because Type IIB restriction enzymes generate fragments long enough so that the unspecified sequences can be confidently identified with the full length sequence of the genomic or cDNA molecule from which each was derived, a “type IIB restriction enzyme tag” or “tag” can serve as a marker for a gene or transcript. A type IIB restriction enzyme tag can be included in a linear oligonucleotide, in a vector or the like.

The term “corresponds” in the phrase “wherein the sequence of said concatemer corresponds to the sequence of at least one transcript which is expressed in the sample” means said sequence is essentially complementary to at least a portion of an RNA transcript present in the sample.

Type IIB Restriction Enzyme Tag Concatemer Libraries (RECORD)

An embodiment of a method of making a nucleic acid library comprising contcatemers of type IIB restriction enzyme tags comprises the steps of: (i) digesting DNA from a biological sample with a type IIB restriction enzyme, (ii) isolating type IIB restriction enzyme tags from the digested DNA, (iii) making the ends of the isolated tags blunt, (iv) ligating the isolated tags into a vector that has blunt ends, wherein the blunt ends of said vector are both flanked by a punctuating restriction enzyme recognition sequence, thereby producing a ligated product, (v) transforming host cells with the ligated product, (vi) isolating the ligated product from the transformed host cells, (vii) digesting the isolated product with a punctuating restriction enzyme, thereby releasing the type IIB restriction enzyme tags, (viii) ligating the type IIB restriction enzyme tags thereby producing concatemers, and (ix) cloning the concatemers, thereby generating a concatenated library comprising type IIB restriction enzyme tags. In one embodiment of making a DNA library comprising contcatemers of type IIB restriction enzyme tags, the Class IIB restriction enzyme is BsaXI.

This method is known as representation by concatenation of restriction digests, or RECORD.

The term “nucleic acid molecule” refers to a nucleic acid of two or more nucleotides. A nucleic acid molecule can be RNA or DNA. For example, a nucleic acid molecule can include messenger RNA (mRNA), transfer RNA (tRNA) or ribosomal RNA (rRNA). A nucleic acid molecule can also include DNA, for example, genomic DNA or cDNA. A nucleic acid molecule can be synthesized enzymatically, either in vivo or in vitro, or the nucleic acid molecule can be chemically synthesized by methods well known in the art. A nucleic acid molecule can also contain modified bases, for example, the modified bases found in tRNA such as inosine, methylinosine, dihyrouridine, ribothymidine, pseudouridine, methylguanosine and dimethylguanosine. Furthermore, a chemically synthesized nucleic acid molecule can incorporate derivatives of nucleotide bases.

The phrase “species of nucleic acid” is defined as any specific nucleic acid.

In an embodiment, the nucleic acid library comprising concatemers of type IIB restriction enzyme tags, the nucleic acid is DNA. The DNA of the instant invention encompasses both cDNA and genomic forms of a gene. “DNA” refers to nucleic acid and encompasses both cDNA and genomic forms of a gene, and an equivalent is RNA or modified DNA or RNA.

A nucleic acid “library” is defined as a plurality of type IIB restriction enzyme tags. A nucleic acid library can encompass 10, 50, 100, 1000, 10,000 type IIB restriction enzyme tags or more. In a preferred embodiment, the nucleic acid library comprises concatemers of type IIB restriction enzyme tags.

A “concatemer” of type IIB restriction enzyme tags is defined as a DNA molecule containing at least two contiguous type IIB restriction enzyme tags that are linked together in sequence. A concatemer may comprise more than 250 type IIB restriction enzyme tags, or from about 1 to 250, or more, type IIB restriction enzyme tags, or 3, 4, 5 or 6 or more contiguous type IIB restriction enzyme tags. In a preferred embodiment, each concatemer is from about 1000 to about 2000 base pairs in length. In one embodiment, the contiguous type IIB restriction enzyme tags found in the concatemer are randomly linked together through a punctuation sequence. “A punctuation sequence” as used herein, means a sequence formed by ligating type IIB restriction enzyme tags in which the two terminal ends of each tag have been digested with a punctuating restriction enzyme. Concatemers allow for efficient sequencing of allowing for efficient sequencing type IIB restriction enzyme tags. Such concatamers are also useful for the analysis of gene expression by identifying the defined nucleotide sequence tag corresponding to an expressed gene in a cell, tissue or cell extract, for example.

The term “biological sample” is defined as any plant, animal or viral material containing nucleic acid. In a preferred embodiment the biological sample is from a vertebrate, preferably a mammal, preferably a human. A biological sample as used herein, is used in its broadest sense, and may comprise a cell, chromosomes isolated from a cell or cell line, genomic DNA, RNA, cDNA, an extract from cells or a tissue or an organ, or a sample suspected of comprising a pathogen.

The phrase “isolating fragments which contain the recognition site of said type IIB restriction enzyme from the digested DNA” comprises any method of isolating those fragments of digested DNA which contain the recognition sequence for the Type IIB restriction enzyme used to digest the DNA. Methods encompassed by the phrase include methods based on size separation by the size. The cleaved DNA fragments can be size-separated and selected using DNA gel electrophoresis. The DNA is electrophoresed through either an agarose or a polyacrylamide matrix. The selection of the matrix will depend on the size of the DNA fragments to be separated. After electrophoresis, the DNA is extracted from the matrix by electroelution, or, if low-melting agarose is used as the matrix, by melting the agarose and extracting the DNA from it.

The phrase “making the ends of the isolated tags blunt” refers to a method of converting the 3′ overhang ends produced by Type IIB endonuclease digestion to blunt ends to make them compatible for ligation. In one embodiment of making the ends of the digested fragments blunt, the DNA is treated in a suitable buffer for at least 15 minutes at 15° C. with 10 units of the Klenow fragment of DNA polymerase I (Klenow) in the presence of the four deoxynucleotide triphosphates. The DNA is then purified by phenol-chloroform extraction and ethanol precipitation.

The phrase “ligating the tags into a vector that has blunt ends” is defined as the method of ligating the purified, blunt ended modified Type IIB digested DNA fragments with a vector that has blunt ends, by combining the DNA fragments with the vector in solution in about equimolar amounts. The solution will also contain ATP, ligase buffer and a ligase such as T4 DNA ligase at about 10 units per 0.5 mg of DNA. The vector may have been treated with alkaline phosphatase or calf intestinal phosphatase. The phosphatasing prevents self-ligation of the vector during the ligation step.

The term “vector” refers to a plasmid, virus or other vehicle known in the art that has been manipulated by insertion or incorporation of the type IIB tagged sequences. Such vectors contain a promoter sequence which facilitates the efficient transcription of the a marker genetic sequence for example. The vector typically contains an origin of replication, a promoter, as well as specific genes that allow phenotypic selection of the transformed cells. Vectors suitable for use in the present invention include for example, pBlueScript (Stratagene, La Jolla, Calif.); pBC, pSL301 (Invitrogen) and other similar vectors known to those of skill in the art. Preferably, the concatemers thereof are ligated into a vector for sequencing purposes. Vectors in which the tagged sequences are cloned can be transferred into a suitable host cell.

“Host cells” are cells in which a vector can be propagated and its DNA expressed. The term also includes any progeny of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

The phrase “transforming host cells with the ligated product” means that vectors in which the tagged sequences are cloned can be transferred into a suitable host cell. “Host cells” are cells in which a vector can be propagated and its DNA expressed. The term also includes any progeny of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl₂ can be used. Transformation can also be performed by electroporation or other commonly used methods in the art.

The term “isolating the ligated product from the transformed host cells” encompasses isolating the DNA using standard techniques, see “Molecular Cloning: A Laboratory Manual”, 2d ed., Cold Spring Harbor Laboratory Press, Sambrook, J., E. F. Fritsch and T. Maniatis eds., 1989. Methods for performing the molecular biology techniques described herein are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

The term “recognition site” of a restriction endonuclease is defined as an area of DNA, which is specifically recognized by a restriction enzyme, and which generally comprises a specific sequence of DNA.

As used herein, the terms “restriction endonucleases” and “restriction enzymes” refer to bacterial enzymes which bind to a specific double-stranded DNA sequence termed a recognition site or recognition nucleotide sequence, and cut double-stranded DNA at or near the specific recognition site. “Type IIP restriction enzymes” is a generic description for all enzymes that recognize symmetric sequences and cleave at symmetrical locations either within the sequence or immediately adjacent to it. Examples of said enzymes include EcorRI, SinI, BglI, and HindII., see Roberts et al. (Nucleic Acids Research, 2003, 31(7):1805-1812).

Other similar endonucleases having at least one recognition site within a DNA molecule (e.g., cDNA) will be known to those of skill in the art (see for example, Current Protocols in Molecular Biology, Vol. 2, 1995, Ed. Ausubel, et al., Greene Publish. Assoc. & Wiley Interscience, Unit 3.1.15; New England Biolabs Catalog, 1995). Any other suitable enzyme known in the art can be used. In addition, restriction endonucleases with desirable properties can be artificially evolved, i.e., subjected to selection and screening, to obtain an enzyme which is useful as a tagging enzyme for the instant invention. Desirable enzymes cut both strands of double-stranded DNA upstream and downstream of their recognition sequences. Artificial restriction endonucleases can also be used. Such endonucleases are made by protein engineering.

Typically, the length of each Type IIB restriction enzyme tag is at least 20 nucleotides, preferably between 27 and 34 nucleotides. In another embodiment, the length of each concatemer has a length from about 1,000 to about 2,000 base pairs but may be smaller or larger. In another embodiment, the library of concatenated tags comprises in general at least 1000 concatemers but may be smaller or larger.

In another embodiment, the restriction tags are generated from a vertebrate, preferably a mammal such as a human, mouse or rat.

Another aspect of the invention comprises a kit, wherein the kit comprises a system for generating a RECORD library of concatemers of Type IIB restriction enzyme tags.

Pathogen Discovery

The invention provides a method that comprises identifying a pathogen in a biological sample. The method comprises generating a RECORD library comprising concatemers of Type IIB restriction enzyme tags, wherein the tags are generated from the biological sample suspected of comprising the pathogen, sequencing the concatenated tags in the library, wherein said tags were generated from the biological sample, identifying a tag whose sequence, or the complement thereof, is not present in a corresponding uninfected or non-diseased sample, and identifying a candidate pathogen by its absence in the reference sample or genome.

The identification of a tag which is not present in a corresponding uninfected or non-diseased sample can be accomplished by computational subtraction against the human genome (Weber et al., 2002, Xu et al., 2003). The term “Computational Subtraction” encompasses a method and a system to detect microbes within a host organism. Computational subtraction provides a method of using a computer system to identify a microbe inhabiting a host organism which comprises the steps of obtaining sequence information from a plurality of sequences from at least one host organism and searching a database of host organism genomic sequences to determine the presence or absence of the plurality of expressed sequences in the database. The absence of at least one of the sequences in the database indicates that the at least one sequence is a candidate microbe sequence. Individual sequences can be searched sequentially; however, preferably, sets of sequences are searched one at a time.

In some cases, the pathogen may be identified by comparison to nucleic acid sequences from known microbial organisms but in other cases further nucleic acid experimentation will be required to identify the novel pathogen. The host organism can be a microorganism, a plant, or an animal, such as a mammal (e.g. human being). The host animal can also be an insect, bird or fish. The biological sample may be genomic double-stranded DNA, single-stranded DNA, messenger RNA or total RNA, each of which may be converted into double-stranded DNA prior to restriction digestion.

A pathogen is defined as any agent which contains nucleic acid and is capable of causing disease in a human, other mammals, or vertebrates. Examples of pathogens include microorganisms such as unicellular or multicellular micro-organisms including but not limited to bacteria, protozoa, fungi, yeast, molds, and mycoplasmas, and non-cellular microorganisms including but not limited to viruses. In a broad sense, the term pathogen can include a symbiotic organism. The pathogen nucleic acid can comprise either DNA or RNA, and this nucleic acid can be single stranded or double stranded. The pathogen may be present in the sample from which the DNA or cDNA was obtained.

The term “computational subtraction”. refers to a method wherein non-human transcripts or genes are detected by sequencing cDNA libraries or genomic libraries from infected tissue and eliminating those transcripts that match the human genome. WO 01/54557A2, by one of the instant inventors, teaches a method for performing computational subtraction to detect microbes within a host organism. This method comprises the steps of obtaining sequence information from a plurality of sequences from at least one host organism and searching a database of host organism genomic sequences to determine the presence or absence of the plurality of expressed sequences in the database. The absence of at least one of the sequences in the database indicates that the at least one sequence is a candidate microbe sequence. Individual sequences can be searched sequentially; however, preferably, sets of sequences are searched at a time.

We have developed a computational subtraction approach to detect microbial causes for putative infectious diseases by filtering a set of human tissue-derived sequences against the human genome. We demonstrate the potential of this method by identifying sequences from known pathogens in established expressed-sequence tag libraries.

This computational subtraction approach can also be used to detect genomic alterations.

Transcript Profiling

The invention provides a method for the detection of the expression of one or more transcripts in a biological sample. This method comprises generation of cDNA from a messenger RNA sample, generating a RECORD library from said cDNA, sequencing at least one or more concatemers comprising Type IIB restriction enzyme tags generated from sample, and comparing the sequence of said tags with the sequence of transcripts from the organism of interest, wherein if the sequence of at least one of the tags corresponds to the sequence of said transcript, then the expression of said transcript in said sample has been detected. This approach also allows the quantification of transcripts by the analysis of tag number and the discovery of novel transcripts within a relevant genome.

Genome Mapping

The invention provides a method of mapping a Type IIB restriction enzyme tag to its location in the genome of an animal, comprising sequencing at least one or more concatemers comprising Type IIB restriction enzyme tags generated from sample the tags in the library generated from a biological sample derived from said animal, wherein at least one of said tags is the tag of interest, and comparing the sequence of a specific tag to markers in the genome of the animal from which the tag was derived, thereby determining the location of a Type IIB restriction enzyme tag in the genome.

Karyotyping

The invention provides a method of identifying one or more regions of deletion, amplification or chromosomal alteration in the genome of an animal, comprising sequencing concatemers comprising Type IIB restriction tags in the library of claim 8, wherein said library is generated from a biological sample derived from said animal, matching the sequence data with precise chromosomal locations, and calculating the density of tags across the chromosome, wherein an increase or a decrease in density is indicative of amplification or deletion, respectively. The invention provides a method of karyotyping using Type IIB restriction enzyme tags can be used to detect gross chromosomal changes.

Concatemer Library Synthesis Kits

In another embodiment, the present invention provides a kit useful for generating RECORD libraries from a DNA sample of interest, including genomic DNA, total DNA or complementary DNA samples. These kits would comprise a detailed protocol for RECORD library construction, two cloning vectors, a shuttle vector for monomer cloning and a destination vector for concatemer cloning, the appropriate type IIB restriction endonuclease, T4 DNA ligase or equivalent, DNA polymerase Klenow fragment or equivalent, other relevant restriction endonucleases (for example Pst I in one embodiment of the invention), and necessary buffers and nucleotides.

To make RECORD libraries from RNA samples, the kit would also incorporate reverse transcriptase, RNase H and DNA polymerase enzymes together with necessary buffers, primers, and nucleotides to make double-stranded DNA from starting RNA.

Experimental Subtraction Methods Using Type IIB Restriction Enyzme Representations (SORT)

The invention also comprises the generation of subtractive libraries using type IIB restriction enzyme tags. In this method, called Subtraction of Restriction Tags (SORT), a collection of type IIB restriction tags is generated from a control nucleic acid population, for example normal human genomic DNA. This restriction tag collection is then immobilized to a solid support such as a bead, a column, a filter, a membrane, or an array.

An independent type IIB restriction tag representation is then generated from an experimental nucleic acid population, for example one generated from a candidate infected tissue or a cancer specimen. This representation is then subtracted by hybridization to the immobilized control representation. The residual DNA is therefore enriched for tags unique to the experimental nucleic acid population. Multiple rounds of enrichment may be carried out if necessary to eliminate tags present in the control.

The representation from the experimental nucleic acid population may be modified with linkers that serve as PCR primers to permit amplification, cloning, concatenation into RECORD libraries, and sequencing. The sequences can then be used for pathogen discovery or genome alteration discovery as described above.

Hybridization Technology Utilizing Type IIB Restriction Enzyme Tags

The invention provides a method for detecting one or more species of nucleic acid in a biological sample by hybridizing a representation from a nucleic acid sample that comprises type IIB restriction enzyme tag to an array composed of single-stranded probes corresponding to one strand of type IIB restriction enzyme tags. In this method, one can generate a representation from a nucleic acid sample that comprises type IIB restriction enzyme tags. This nucleic acid sample may by a genomic DNA sample, another DNA sample, a complementary DNA sample generated by reverse transcription of RNA, a DNA sample prepared by whole genome amplification, or a DNA sample comprising a RECORD library. The method consists of digesting the DNA sample with a type IIB restriction endonucleases, purifying the digested tags, hybridizing single strands of said isolated tags to a microarray containing nucleic acid oligomeric probes, and detecting hybridization of said tags to the nucleic acid probes on said microarray, thereby detecting said one or more species of nucleic acid in said sample. In one embodiment of the method, the sample is a library which comprises the concatemer tags that was generated from DNA or RNA of a biological sample. In another embodiment of the invention, the probes consist of a length identical to the length of said tags. In another embodiment of the method, said tags are labeled. In another embodiment of the invention, the probes on the solid surface consist of a length of between 27 and 32 base pairs. In another embodiment of the invention, the probes on the solid surface are selected from the group consisting of Type IIB restriction enzyme tags. In another embodiment of the invention, the probes on the solid surface target only type IIB restriction endonuclease cleavage products which are unique in the genome. In another embodiment of the invention, the probes on the solid surface target only type IIB restriction endonuclease cleavage products which are absent from the genome, to detect pathogen sequences. In another embodiment of the invention, the solid surface comprises a chip, a bead, derivatized glass, or silicon, or nylon or nitrocellulose, for example.

The term “species” is defined herein in a broad sense, and encompasses any of the numerous types of nucleic acid, either synthetic or naturally derived f from a biological sample, wherein said biological sample may be of recombinant origin. Examples of a species include a spliced or unspliced mRNA, hRNA, gene, ribozyme, transfer RNA, ribosomal RNA, nucleic acid endogenous or exogenous to the cell, fragments thereof.

The term “array”, “microarray”, “microarray of molecules” or “array of molecules” refers to a plurality of molecules stably bound to a solid support. An array can comprise, for example, nucleic acid, oligonucleotide or polypeptide-nucleic acid molecules. It is understood that, as used herein, an array of molecules specifically excludes molecules that have been resolved electrophoretically prior to binding to a solid support and, as such, excludes Southern blots, Northern blots and Western blots of DNA, RNA and proteins, respectively.

“Microarrays”, useful in the identification of differentially expressed nucleic acid sequences, may be any microarray known in the art that comprises defined sequences. A polynucleotide microarray refers to a plurality of unique nucleic acids probes, attached to one surface of a solid support at a density exceeding 20 different nucleic acids/cm² wherein each of the nucleic acid probes is attached to the surface of the solid support in a non-identical preselected region. Because the nucleic acid probes are in known positionally distinct orientations on the substrate, one need only examine the hybridization pattern of a target oligonucleotide on the substrate to determine the sequence of the target oligonucleotide. Use and preparation of these arrays for hybridizing with is generally described in PCT patent publication Nos. WO 92/10092, WO 90/15070, U.S. patent application Ser. Nos. 08/143,312 and 08/284,064. Each of these references is hereby incorporated by reference in its entirety for all purposes.

In one embodiment, the nucleic acid attached to the surface of the solid support is DNA. In a preferred embodiment, the nucleic acid attached to the surface of the solid support is cDNA. In another preferred embodiment, the nucleic acid attached to the surface of the solid support is cDNA synthesized by polymerase chain reaction (PCR). In another embodiment, the nucleic acid attached to the surface of the solid support comprises ESTs. In a preferred embodiment, the nucleic acid attached to the surface of the solid support comprises Type IIB restriction enzyme tags. In another embodiment, the nucleic acid attached to the surface of the solid support comprise RNA. Preferably, the nucleic acid attached to the surface of the solid support as an array, according to the invention, is at least 20, nucleotides in length. Preferably, a nucleic acid comprising an array is less than 6,000 nucleotides in length. More preferably, a nucleic acid comprising an array is less than 500 nucleotides in length. In one embodiment, the array comprises at least 500 different nucleic acids attached to one surface of the solid support. In another embodiment, the array comprises at least 10 different nucleic acids attached to one surface of the solid support. In yet another embodiment, the array comprises at least 10,000 different nucleic acids attached to one surface of the solid support.

Through the use of these oligonucleotide arrays, the specific hybridization of a target sequence(s) can be tested against a large number of individual probes in a single reaction. Such oligonucleotide arrays employ a substrate, comprising positionally distinct sequence specific recognition reagents, such as polynucleotides, localized at high densities.

“Label” is defined as radioisotope, a fluorescent compound, a bioluminescent compound, a chemiluminescent compound, a metal chelator, or an enzyme label.

The present application also includes solid surfaces with arrays of Type IIB restriction enzyme tags, wherein the tags of said libraries are attached to a solid surface, wherein the solid surface comprises a chip, a bead, derivatized glass, or silicon.

The concatemer libraries of the instant invention can comprise Type IIB tagged genomic sequences as well as Type IIB tagged cDNA sequences. A preferred embodiment of this invention is a chip that contains nucleic acid probes comprising type IIB restriction enzyme tagged DNA sequences.

The term oligonucleotides: An oligonucleotide is a single-stranded DNA or RNA molecule, typically prepared by synthetic means. Alternatively, naturally occurring oligonucleotides, or fragments thereof, may be isolated from their natural sources or purchased from commercial sources. Those oligonucleotides employed in the present invention will be 4 to 100 nucleotides in length, preferably from 6 to 30 nucleotides, although oligonucleotides of different length may be appropriate. Suitable oligonucleotides may be prepared by the phosphoramidite method described by Beaucage and Carruthers, Tetrahedron Lett., 22:1859-1862 (1981), or by the triester method according to Matteucci et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exists in a hydrogen-bonded, helical array typically associated with, for example, DNA. In addition to the 100% complementary form of double-stranded oligonucleotides, the term “double-stranded” as used herein is also meant to refer to those forms which include such structural features as bulges and loops, described more fully in such biochemistry texts as Stryer, Biochemistry, Third Ed., (1988), previously incorporated herein by reference for all purposes.

A second preferred method for making microarrays is by making high-density oligonucleotide arrays, as disclosed in Rosetta's U.S. Pat. No. 6,218,122. U.S. Pat. No. 6,218,122 discloses that techniques are known for producing arrays containing thousands of oligonucleotides complementary to defined sequences, at defined locations on a surface using photolithographic techniques for synthesis in situ (see, Fodor et al., 1991, Light-directed spatially addressable parallel chemical synthesis, Science 251:767-773; Pease et al., 1994, Light-directed oligonucleotide arrays for rapid DNA sequence analysis, Proc. Natl. Acad. Sci. USA 91:5022-5026; Lockhart et al., 1996, Expression monitoring by hybridization to high-density oligonucleotide arrays, Nature Biotech 14:1675; U.S. Pat. Nos. 5,578,832; 5,556,752; and 5,510,270, each of which is incorporated by reference in its entirety for all purposes) or other methods for rapid synthesis and deposition of defined oligonucleotides (Blanchard et al., 1996, High-Density Oligonucleotide arrays, Biosensors & Bioelectronics 11: 687-90). When these methods are used, oligonucleotides (e.g., 20-mers) of known sequence are synthesized directly on a surface such as a derivatized glass slide. Usually, the array produced is redundant, with several oligonucleotide molecules per RNA. Oligonucleotide probes can be chosen to detect alternatively spliced mRNAs. Another preferred method of making microarrays is by use of an inkjet printing process to synthesize oligonucleotides directly on a solid phase, as described, e.g., in copending U.S. patent application Ser. No. 09/008,120 filed on Jan. 16, 1998 by Blanchard entitled “Chemical Synthesis Using Solvent Microdroplets”, which is incorporated by reference herein in its entirety.

One embodiment of oligonucleotide synthesis on an array used by Affymetrix and disclosed in U.S. Pat. No. 5,837,832 is the VLSIPS method. In the VLSIPS method, light is shone through a mask to activate functional (for oligonucleotides, typically an —OH) groups protected with a photoremovable protecting group on a surface of a solid support. After light activation, a nucleoside building block, itself protected with a photoremovable protecting group (at the 5′-OH), is coupled to the activated areas of the support. The process can be repeated, using different masks or mask orientations and building blocks, to prepare very dense arrays of many different oligonucleotide probes.

Other methods for making microarrays, e.g., by masking (Maskos and Southern, 1992, Nuc. Acids Res. 20:1679-1684), may also be used. In principal, any type of array, for example, dot blots on a nylon hybridization membrane (see Sambrook et al., Molecular Cloning—A Laboratory Manual (2nd Ed.), Vol. 1-3, Cold Spring Harbor Laboratory, Cold Spring Harbor, N.Y., 1989, which is incorporated in its entirety for all purposes), could be used, although, as will be recognized by those of skill in the art, very small arrays will be preferred because hybridization volumes will be smaller.

One embodiment of oligonucleotide synthesis on an array is used by NimbleGen™ as described by McCormick, M. in Innovations in Pharmaceutical Technology 3(11), 88-93 (2003). NimbleGen builds custom, high-density microarrays based on its proprietary Maskless Array Synthesizer (MAS) technology. At the heart of the MAS technology is the Digital Micromirror Device (DMD). The DMD is an array of 786,000 tiny aluminum mirrors, arranged on a computer chip, where each mirror is individually addressable. Using these tiny aluminum mirrors to shine light in specific patterns, coupled with the photo deposition chemistry, produces arrays of oligonucleotide probes. The DMD patterns light by flipping mirrors on and off according to the instruction in a “digital mask” file. The arrays are synthesized on a standard 25 by 75 mm glass microscope slide compatible with commercial array scanners. In an individual synthesis cycle, an incoming photoprotected phosphoramidite is coupled to the hydroxyl-terminal 5′ end of an individual oligonucleotide in the presence of an activator. The linkage is stabilized by a brief oxidation step, leaving a 5′ photoprotected olgonucleotide. When a subsequent nucleotide addition is required at this location, the photoprotecting group is cleaved by a brief exposure to ultraviolet light, liberating a free 5′ hydroxyl group for the next round of replication. A commercially available synthesis instrument controls the delivery of DNA synthesis chemistry.

These DNAs can be obtained by, e.g., polymerase chain reaction (PCR) amplification of gene segments from genomic DNA, cDNA (e.g., by RT-PCR), or cloned sequences.

An alternative means for generating the nucleic acid for the microarray is by synthesis of synthetic polynucleotides or oligonucleotides, e.g., using N-phosphonate or phosphoramidite chemistries (Froehler et al., 1986, Nucleic Acid Res 14:5399-5407; McBride et al., 1983, Tetrahedron Lett. 24:245-248). Synthetic sequences are between about 15 and about 500 bases in length, more typically between about 20 and about 50 bases. In some embodiments, synthetic nucleic acids include non-natural bases, e.g., inosine. As noted above, nucleic acid analogues may be used as binding sites for hybridization. An example of a suitable nucleic acid analogue is peptide nucleic acid (see, e.g., Egholm et al., 1993, PNA hybridizes to complementary oligonucleotides obeying the Watson-Crick hydrogen-bonding rules, Nature 365:566-568; see also U.S. Pat. No. 5,539,083).

For a description of hybridization of nucleic acids to solid supports, see U.S. Pat No. 5,800,992 incorporated by reference herein. Hybridization conditions: Stringent hybridization conditions will typically include salt concentrations of less than about 1 M, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

### BRIEF DESCRIPTION OF THE DRAWINGS

FIG. 1 An example of protocol for construction of a concatenated library of BsaXI tags. Primary transformation is here done using PUC19 vector (Invitrogen) that has been modified to contain two PstI sites flanking an EcoRV site. Secondary cloning is done using PstI-cleaved pZErO-1 (Invitrogen).

FIG. 2. Table of Type IIB restriction enzymes.

FIG. 3. Digital Karyotyping of chromosome 9 from cell line HCC38 using concatenated library of BsaXI tags. Deletion is indicated by low density of tags found around virtual tag 8000.

FIG. 4. Probes where signal intensities were quantified on array. BioU are biotinylated uracils for labeling and | represents the surface on the array.

FIG. 5. Signal intensities from BsaXI probes sites on the Affymetrix 10K SNP array after hybridization with BsaXI tags and control DNA. Mismatch probes have the central base altered.

### DETAILED DESCRIPTION OF THE INVENTION

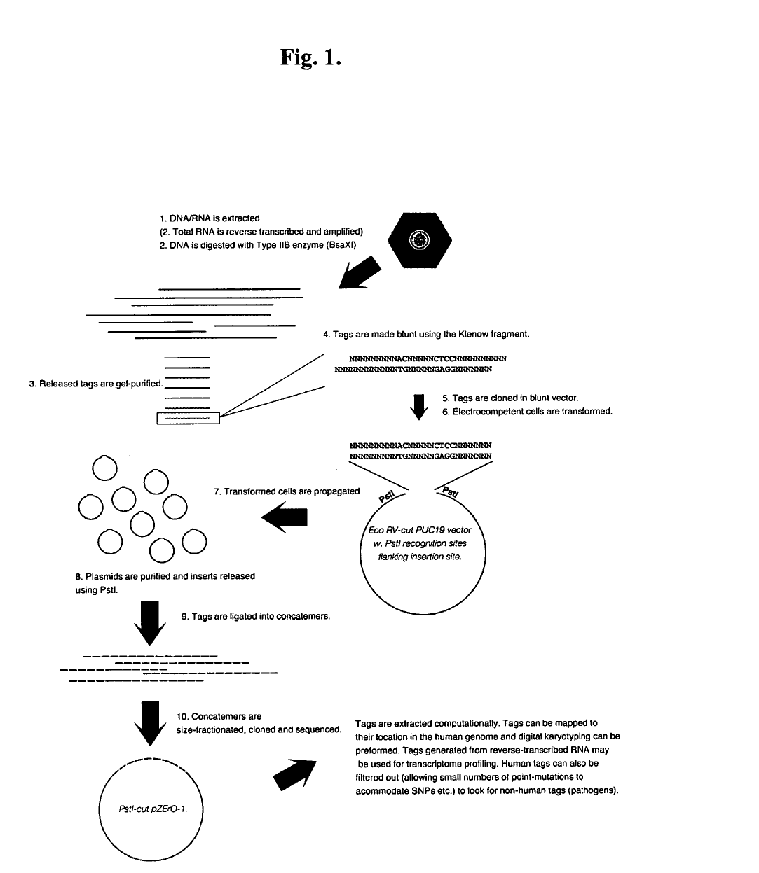

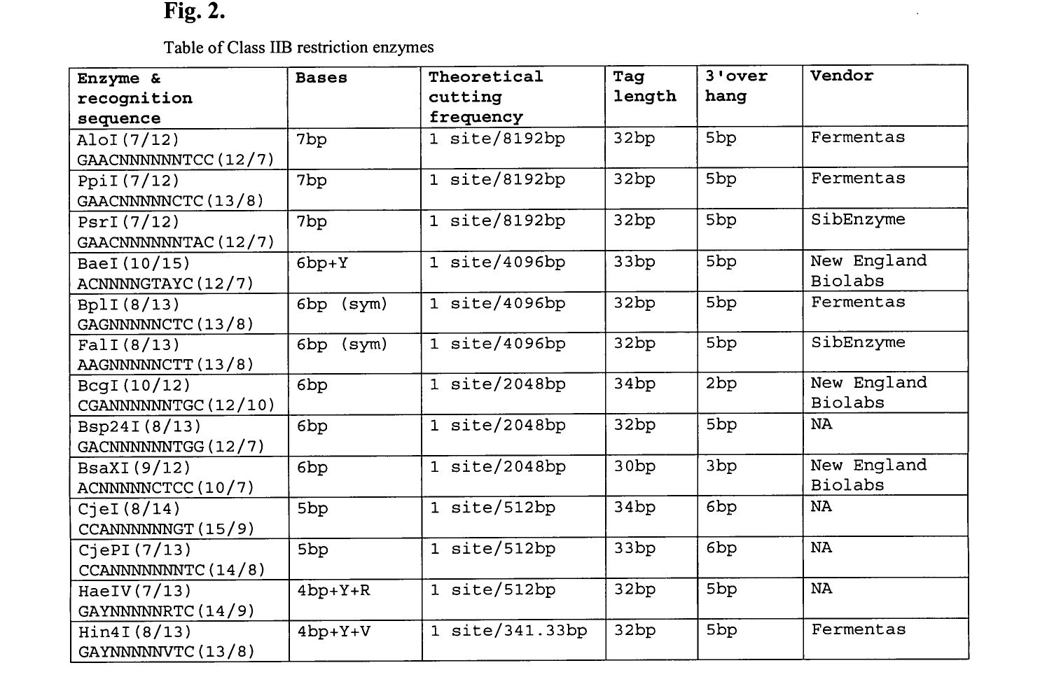

The invention centers on generating tags from DNA digested with a Type IIB restriction enzyme. Type IIB restriction enzymes are defined as site-specific endonucleases that cut both strands of double-stranded DNA upstream and downstream of their recognition sequences (Roberts et al. 2003, FIG. 1). Different members of this group of enzymes release fragments of a specific size, and they have unique recognition sequences. Known examples of Type IIB restriction enzymes are; AloI, PpiI, PsrI, BaeI, BplI, FalI, BcgI, Bsp24I, BsaXI, CjeI, CjePI, HaeIV and Hin4I. For example, the recognition site of CjeI is (8/14)CCANNNNNNGT(15/9) which indicates that cleavage occurs 8 bases in front of the sequence on the strand written and 14 bases before the sequence on the complementary strand, and that cleavage also occurs 15 bases after the recognition sequence on the strand written and 9 bases after the sequence on the complementary strand yielding a tag length of 34 base pairs. The recognition sites and cleavage sites of several Type IIB restriction enzymes are listed in FIG. 2.

Libraries Comprising Concatemers of Type IIB Restriction Enzyme Tags

The present invention provides a simple method for construction of libraries comprising DNA Type IIB restriction enzyme concatenated tags. In a preferred embodiment of the invention, the method only includes two cloning steps, and does not include a PCR step that is prone to introduce mutations in the sequence of the tags.

The main problem in constructing concatenated libraries of Type IIB restriction enzyme tags, is because that the cohesive ends of the Type IIB restriction enzyme tags that are generated upon digestion with a type IIB restriction enzyme have an unknown composition. Type IIB restriction enzymes will release tagged DNA fragments by cutting substrate DNA at fixed distances upstream and downstream of the recognition sequences, regardless of the sequence in the actual cut site. For example, the type IIB restriction enzyme BsaXI will release tags of the form:

| | |
|---|---|
| 5′NNNNNNNNNACNNNNNCTCCNNNNNNNNNN3′ | (SEQ ID NO:1) |
| 3′NNNNNNNNNNNNTGNNNNNGAGGNNNNNNN5′ | (SEQ ID NO:2) |

To circumvent this problem, the recessive ends are filled in, or the protruding ends are blunted. Since all Type IIB enzymes described thus far leave 3′ overhangs, the only possibility is blunting the protruding ends, (FIG. 1).

If the starting material for generating the library is RNA, the first step will be to reverse transcribe and amplify either total or polyadenylated RNA in order to generate cDNA. cDNA may be prepared according to the following method. Total cellular RNA is isolated (as described) and passed through a column of oligo(dT)-cellulose to isolate polyA RNA. The bound polyA mRNAs are eluted from the column with a low ionic strength buffer. To produce cDNA molecules, short deoxythymidine oligonucleotides (12-20 nucleotides) are hybridized to the polyA tails to be used as primers for reverse transcriptase, an enzyme that uses RNA as a template for DNA synthesis. Alternatively, mRNA species are primed from many positions by using short oligonucleotide fragments comprising numerous sequences complementary to the mRNA of interest as primers for cDNA synthesis. The resultant RNA-DNA hybrid is converted to a double stranded DNA molecule by a variety of enzymatic steps well-known in the art (Watson et al., 1992, Recombinant DNA, 2nd edition, Scientific American Books, New York).

If the starting material is DNA, the Type IIB digest can be done directly. After digestion of the cDNA or DNA, the Type IIB Restriction Enzyme tags are blunted using the Klenow fragment. After blunting, the tags are ligated into a vector that has been opened using a bunt-cutting enzyme (such as EcoRV). The vector is modified to contain identical recognition sequences for a restriction enzyme with a 6 (or 8) base pair punctuation recognition sequence (such as PstI) immediately flanking the blunt-cutter recognition site. The ligation is transformed into highly competent E. coli cells, which can then be propagated either on agar-plates or in liquid culture. After incubation, the cells can be collected, and their plasmids purified. Inserts are released using the appropriate punctuating restriction enzyme (PstI), and purified using acrylamide gel-electrophoresis. The purified tags have compatible cohesive ends and can be ligated into concatenates. These concatenates can be size-fractionated and large concatenates can be cloned in a library, and sequenced. The concatenated tags from the library can be sequenced by standard methods (see for example, Current Protocols in Molecular Biology, supra, Unit 7) either manually or using automated methods. Sequence information from individual tags is then extracted computationally and a database is made. This database can then be analyzed in several different ways:

An advantage of libraries comprising DNA Type IIB restriction enzyme concatenated tags is that the degree of resolution can be specified. Dependent upon what Type IIB enzyme is used, the average distance between cut-sites can be anywhere from about 10 kb to <500 base pairs (assuming no base compositional bias).

An advantage of this innovative method of making a library comprising DNA Type IIB restriction enzyme concatenated tags is that the integrity of the tags is very well preserved, giving sets of tags in which more than 90% of them were identical to their corresponding sequence in the starting material. The high fraction of tags that stay intact throughout the library making process and can thus be mapped and analyzed means that more useful data can be generated from fewer sequencing reactions.

For sensitive pathogen discovery and efficient karyotyping, it is imperative that the libraries comprising DNA Type IIB restriction enzyme concatenated tags used are of high complexity. The pool of concatenated tags available for sequencing must approximate the diversity of tags generated by the initial digest. To ensure this, all of our primary transformations used for library making were estimated to contain more than 10⁸ clones based on colony counts. We also did multiple-recapture calculations of final tag databases to calculate theoretical complexity of tags in concatenates (Darroch 1958), and our karyotyping libraries comprising DNA Type IIB restriction enzyme concatenated tags had complexities close to that estimated from tags generated by computational digests of the human genome.

We have made high complexity libraries comprising Type IIB restriction enzyme tags by using a large amount of starting material, <2 ug of DNA. The diversity of the tags is easily maintained throughout the protocol of making the library.

Alternatively, libraries can be made from nanogram amounts of DNA using the method of whole genome amplification (WGA) of starting material. We have demonstrated that WGA can give enough material for direct formation of concatenates that can be cloned even without a primary transformation. This extremely fast protocol makes it possible to make concatenated libraries from small amounts of DNA in 2-3 days. WGA has been shown to have a slight bias in synthesis of certain parts of the human genome, but this differential amplification of certain genomic loci seems highly reproducible (Lage et al. 2003). As with any karyotyping project based on analyses of whole genome amplified DNA, results will thus have to be normalized to accommodate this sequence representational bias.

Transcript Profiling

A) Transcript Profiling Using a Library Comprising Concatemers of Type IIB Restriction Enzyme Tags

The present invention provides for a method for transcript profiling using a library comprising concatemers of Type IIB Restriction Enzyme tags using Type IIB restriction enzyme tags from a Type IIB Concatenated Library that has been generated from DNA, wherein the DNA is cDNA prepared from RNA obtained from a sample. An aspect of the invention is a method for the detection of expression of a transcript in a sample. In this aspect, the RNA is reverse transcribed into cDNA, digested with a Type IIB restriction enzyme, generating Type IIB restriction enzyme tags. These tags represent fragments of transcripts present in the sample. The method comprises sequencing at least one concatemer found in a concatenated Type IIB restriction enzyme tag library, wherein the DNA of the library is cDNA prepared from the RNA isolated from said sample, and wherein the sequence of said concatemer corresponds to the sequence of at least one transcript which is expressed in the sample. The identity of the transcripts that the tags represent can be discovered by comparing the nucleotide sequences of the tags to sequence databases of the organism from which the sample was derived. This comparison allows the identification of activated genes and could also potentially aid in the identification of novel transcripts.

The transcript, or set of transcripts to be identified can be expressed in a sample in response to any number of processes, including immune and autoimmune processes, normal and abnormal developmental processes, normal and abnormal homeostatic processes. The transcript, or set of transcripts to be identified can be expressed in a sample as a result of in any number of responses, including responses to a disease or disorder, to external or internal stimuli.

B) Transcript Profiling Using Hybridization of Type IIB Restriction Enzyme Tags

In another aspect of the invention, the identity of transcripts that the tags represent can be discovered by hybridizing a plurality of non-concatenated Type IIB restriction enzyme tags to oligonucleotides on a solid surface. In one embodiment, the identity of transcripts that the tags represent can be discovered by hybridizing a plurality of non-concatenated Type IIB restriction enzyme tags to an array of oligonucleotides whose position and identity on a chip are predetermined.

Precise hybridization conditions can be achieved because of the uniformity of length of the Type IIB restriction enzyme tags that hybridize to nucleotides on a solid surface produce more accurate results. Type IIB restriction enzyme tags produced upon digestion with a single Type IIB restriction enzyme are uniform in length because a Type IIB restriction enzyme cuts the DNA at a defined length both upstream and downstream of its recognition site. The length of a type IIB restriction enzyme tag is typically in the range of about 27-34 base pairs, depending on the specific Type IIB restriction enzyme. The length of the Type IIB restriction enzymes tags generated by each Type IIB restriction enzyme varies in length with respect to the respective recognition sequence of the Type IIB restriction enzyme used, due to the number of the base pairs flanking the restriction recognition site and due to the number of base pairs in the recognition site. The uniformity in the length of the fragments generated by digestion with any single Type IIB restriction enzyme allows the use of these fragments as tags in precise hybridization conditions. Accordingly, the present invention provides a method wherein the identity and relative quantity of the transcripts that the Type IIB restriction enzyme tags represent is determined by hybridizing non-concatenated Type IIB restriction enzyme tags, with oligonucleotides immobilized on a solid support (e.g., a chip, derivatized glass, or silicon, or nylon or nitrocellulose) wherein the sequence of each oligonucleotide and its position on the solid support is predetermined. The solid support is then used to determine differential expression of the tags contained within that support (e.g., on a grid on a chip) by hybridization of the oligonucleotides on the solid support with tags produced from cells under different conditions (e.g., different stage of development, growth of cells in the absence and presence of a growth factor, normal versus transformed cells, comparison of different tissue expression, etc).

Many commercially available microarray platforms have probes that are in the size-rang of a Type IIB restriction enzyme tag, and it is thus possible to design an array where experimentally tags can be hybridized to a microarray very effectively with high degree of specificity. Microarrays have been applied successfully for methods of gene discovery, monitoring gene expression patterns, detecting mutations and polymorphisms, and mapping of genomic clones (Guo et al. Genome Research, (2001)12:447-457, Pease (1994) PNAS 91:5022-26, Lipshutz et al. (1995) Biotechniques 19:442-7, Lockhart et al. (1996) Nat. Biotechnology 14:1675-80, Sapolsky and Lipshutz (1996)Genomics 33:445-456), incorporated by reference.

Specifically, tags generated by Type IIB restriction enzyme digestion of DNA or reverse transcribed RNA have a uniform size and can be extracted from acrylamide gels in almost pure form. Dependent on what enzyme is used to do the digest, the length of the single strands can be anywhere from about 27 to 34 base pairs. Gel-purified tags can be labeled using standard terminal-transferase protocols and hybridized directly to an array. The labeled or unlabeled tags can be separated into single-stranded molecules which are preferably serially diluted and added to a solid support (e.g., a silicon chip as the Affymetrix 10K SNP array or other chip described by Fodor, et al., Science, 251:767, 1991) containing oligonucleotides representing single nucleotide polymorphisms. The tags can be labeled using standard terminal-transferase protocols and hybridized directly to an array. Furthermore, the transcript abundance of the sample can be estimated based on the density of the tags that hybridize to an array.

In one embodiment, the present invention encompasses these microarray based methods which use individual non-concatenated Type IIB restriction enzyme tags to hybridize to a microarray containing nucleic acid oligonucleotide probes on the microarray, whereby the hybridization of the tags allows the identification and quantification of the species of nucleic acid represented by the hybridized tag.

In this case that species would be RNA. However, depending on the nature (cDNA vs. genomic DNA for example) both transcriptome and genome profiling can be accomplished, as well as techniques designed to subtract out unwanted species from a sample.

Pathogen Discovery

A. Pathogen Discovery Using a Library Comprising Concatemers of Type IIB Restriction Enzyme Tags

The present invention provides a method for detecting the presence of foreign DNA in a biological sample using a library comprising concatemers of Type IIB Restriction Enzyme Tags, which were generated from the sample. The “foreign” DNA encompasses DNA not normally or typically present in the biological samples. In one embodiment of the invention, the biological sample is of human origin and the foreign DNA is derived from a human pathogen. The method encompasses generating a library of concatemers of Type IIB Restriction Enzyme Tags from a sample suspected of containing a pathogen, sequencing the concatemers, and comparing the obtained sequences to sequences obtained from pathogen free samples. When the sample is of human origin, then tags that contain DNA that corresponds to non-human DNA are identified by analyzing the sequences of those tags which have no apparent matches to the human genome. Accordingly, an embodiment of this invention presents a method of identifying a pathogen in a sample using a library comprised of concatenated Type IIB restriction enzyme tags generated from DNA or RNA obtained from a pathogen. Sequencing the tags in the library and identifying the tags which are not present in a corresponding uninfected or non-diseased sample, allows the identification of the nucleotide sequence of the pathogen by comparison to nucleic acid databases containing sequences derived from pathogens.

A pathogen is defined as any agent which contains nucleic acid and is capable of causing disease in a human, other mammals, or vertebrates. Examples of pathogens include microorganisms such as unicellular or multicellular microorganisms including but not limited to bacteria, viruses, protozoa, fungi, yeast, molds, and mycoplasmas. The pathogen can comprise either DNA or RNA, and this nucleic acid can be single stranded or double stranded. The pathogen may be present in the sample from which the DNA or cDNA was obtained. This method can be used to identify novel pathogens in disease that appear infectious, but where the pathogen has not yet been characterized.

For pathogen discovery projects, it is imperative that sequence tags stay as intact as possible from starting material to sequenced concatenates. The sequence integrity of the tags in the Type IIB restriction enzyme concatemer library generated by the instant method is higher than tags generated by methods that require a PCR amplification step. Novel pathogens can be discovered based on the presence on ‘non-human’ tag sequences (Weber et al. 2002, Xu et al. 2003). These non-human tag sequences are determined using software that is capable of subtracting out tags that match the human genome very efficiently and accurately. This effectiveness in subtracting out tags with human sequences would be reduced if the tags have a significant number of alterations in their sequences.

The premise of the software is that it subtracts out tags that match the human genome by filtering away tags that are perfect matches, filtering tags with a sequence that is one base different from the human genome, and filtering away tags that are a perfect match to a sequence in the human genome after said tags have been trimmed away by one base on either end of the tag. The remaining tags are considered non-human, foreign material, and compared to pathogenic sequences. The foreign material may then be analyzed as being derived from a pathogen. Without the subtraction technology, tens of thousands of sequencing reactions may be required to identify Type IIB restriction enzyme tags that contain pathogenic sequences.

B. Pathogen Discovery Using Hybridization of Type IIB Restriction Enzyme Tags

The present invention provides a method for determining the identity and quantity of a oligonucleotide species in a sample suspected of comprising a pathogen, or other foreign DNA or RNA. Type IIB restriction enzyme tags that were generated from samples suspected of comprising a pathogen said pathogenic sequences present in a sample. In a preferred embodiment, the single tags are generated by digesting the tags from concatemers of Type IIB Restriction enzyme tags linked in a library of said concatemers.

The Type IIB restriction enzyme tags are then hybridized to a solid support wherein said solid support contains pathogenic derived oligonucleotide probes of defined sequence and position on the solid support. In a preferred embodiment of the instant invention, the solid support is a chip and the oligonucleotides are positioned in a microarray. In a preferred embodiment, the length of the probes on the chips is approximately 20-50 nucleotides, preferably 27-34 nucleotides in length. Arrays that contain pathogenic oligonucleotide probes are well known in the art, as illustrated by the viral detection microarray taught by Wang et al. (2002) PNAS 99:15687. The viral detection array contains 1600 unique viral oligonucleotide probes derived from approximately 140 distinct viral genomes. Because the sequences of the probes were generated using the most highly conserved sequences within each viral family, these probes can be used to identify new viral species as well as identify viral subtypes in a biological sample.

The Type IIB restriction enzyme tags are hybridized under conditions of high specificity due to the uniformity of length of the tags, and their similar length relative to the length of the probes on the chip. The high specificity, microarray based strategy for pathogen detection can be used in methods of diagnosis of pathogen based disorders and also in methods of discovery of new pathogens, and new micro-organisms.

Subtraction Technology

An aspect of the invention is a method to reduce the complexity of Type IIB tagged sequences generated from an experimental sample, wherein the method comprises separately generating type IIB tagged sequences from an experimental sample and from a control sample, labeling said Type IIB tagged tags generated from the control sample, attaching the labeled tags to a solid surface, hybridizing the experimentally generated tags. In a preferred embodiment, the solid surface is selected from the group consisting of a chip, a bead, derivatized glass, or silicon.

A microarray can be used to subtract tags generated by Type IIB digestion. This normalization/subtraction approach can be used by itself to analyze individual tags, or can be also combined with concatenation protocols. Briefly, tags that represent a collection of targets you want to normalize your library against or eliminate/reduce abundance of can either be synthesized in vitro or generated by digestion of DNA from control specimen. The tags can then be attached to a solid phase (magnetic beads, resin, nitrocellulose, chip etc.). Experimentally generated tags can then be hybridized to the attached probes, and unwanted tags can be physically subtracted out, or the library normalized. Unbound tags can then be further analyzed. As described for the array approach, we believe that the uniformity in length and the high purity of the tags will allow highly efficient subtractions as well as normalization.

Microarray Comprising Type IIB Restriction Enzyme Tagged DNA Sequences

Also encompassed by this invention is a method to make a chip that contains nucleic acid probes comprising Type IIB restriction enzyme tagged DNA sequences.

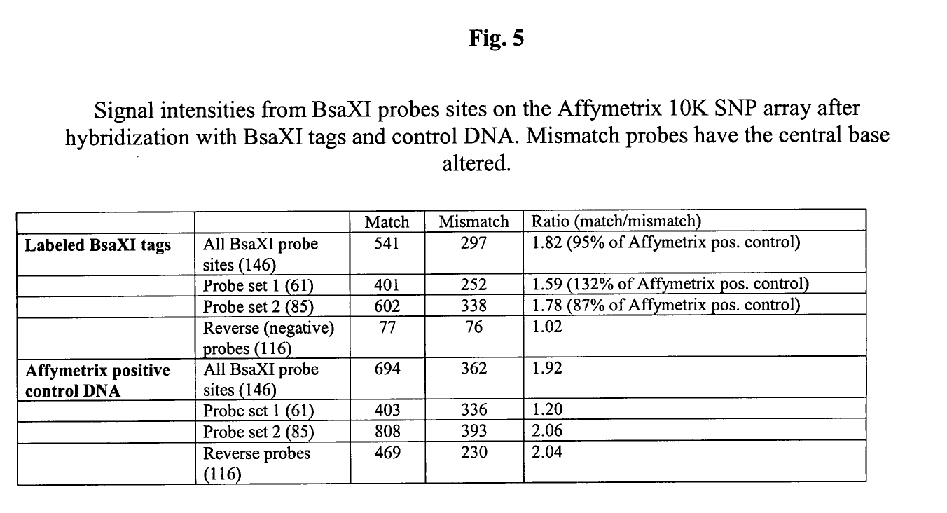

Arrays based on Type IIB tags will represent an ideal situation for specific and effective hybridization between tags and probes. There will be a very low background of labeled DNA present in the hybridization reaction that is not complementary to any of the probes on the array. Arrays can be made where the probes are of the same length as the tags, making the hybridization reaction optimal. The level of background signal should also be even lower than indicated by the signal/noise ratios in the table of FIG. 5. Most tags on a Type IIB tag array will most likely be more than a single base different from each other, decreasing chances of non-specific hybridization of tags.

Construction of a Microarray

In a microarray, nucleic acids (e.g., oligonucleotides or cDNA) are attached to a substantially solid support. In a preferred embodiment, the substantially solid support to which the nucleic acids are attached is a supporting film or glass substrate such as a microscope slide. A nucleic acid microarray containing Type IIB restriction tags as an array of probes according to the invention was constructed as follows, and may be fabricated on the substrate according to the pioneering techniques disclosed in U.S. Pat. No. 5,143,854 or International Publication No. WO 92/10092, which are hereby incorporated by reference. The combination of photolithographic and fabrication techniques may, for example, enable each probe sequence (“feature”) to occupy a very small area (“site”) on the support. In some embodiments, this feature site may be as small as a few microns or even a single molecule. For example, about 10⁵ to 10⁶ features may be fabricated in an area of only 12.8 mm² Companies presently manufacturing and marketing oligonucleotide or cDNA microarrays include Affymetrix, Santa Clara, Calif.; ClonTech, Palo Alto, Calif.; Corning, Inc., Corning, N.Y.; and Motorola, Inc., BioChip Systems Division, Northbrook, Ill. See U.S. Patent Application No. 20030044823

If an Type IIB restriction enzyme with a high cutting frequency is chosen (for instance BsaXI, where the average spacing between unique tags in the human genome is approximately 3000 bases), this will make it possible to make arrays with a unprecedented resolution. Only limited by the density of which probes can be synthesized on an array, this high resolution approach to Karyotyping and gene expression profiling can be used to detect small regions of amplification, heterozygous or homozygous deletions and to discover novel transcripts. Hybridization of RNA-derived tags to a genomic tag array could potentially identify novel genes.

Use of Chips Containing Type IIB Restriction Enzyme Tags as Probes

As described above, the chips containing Type IIB Restriction Enzyme Tags as Probes can be used in a method for the detection of expression of a transcript in a sample, or to detect foreign nucleic acid in a sample, such as pathogenic DNA, or to subtract out unwanted nucleic acid molecules from a sample, or to purify select nucleic acid molecules from a sample. In one embodiment, Type IIB restriction enzyme tags can be used as tags to hybridize to said chips containing Type IIB Restriction Enzyme Tags as Probes.

Alternatively, the identity of the transcripts that the Type IIB restriction enzyme tags represent can be discovered by hybridizing the tags, or concatemers of said tags, with oligonucleotides immobilized on a solid support (e.g., nitrocellulose filter, glass slide, silicon chip) wherein the sequence of each oligonucleotide and its position on the solid support is known. The labeled or unlabeled tags can be separated into single-stranded molecules which are preferably serially diluted and added to a solid support (e.g., a silicon chip as the Affymetrix 10K SNP array. or other chip described by Fodor, et al., Science, 251:767, 1991) containing oligonucleotides probes. Gel-purified tags can be labeled using standard terminal-transferase protocols and hybridized directly to an array.

Karyotyping

The invention describes methods of Karyotyping using Type IIB restriction enzyme tag sequences to detect gross chromosomal changes such as fusions, as well as amplifications and deletions of specific loci. Such changes often are associated with cancer and other disorders such as Downs syndrome. DNA or cDNA is obtained from a normal sample or a sample suspected of having a chromosomal abnormality, digested with a Type IIB restriction enzyme, generating Type IIB restriction enzyme tags. The tags are concatenated and sequenced. The sequence data is computationally matched the with precise chromosomal locations, as described in Wang et al (PNAS) 99(25):16156-16161.

In one embodiment of the invention, a sliding window will works as follows: using a computer a virtual digest of the human genome is performed, which maps all potential tags in the genome (for instance BsaXI tags). Then, actual concatenated tags are sequenced and mapped to their locations in the genome on the basis of their sequence. The virtual tags and the sequenced tags are then compared. A reference region of a fixed size (based on an arbitrary number of virtual tags) is chosen, for example 1000 virtual tags, which in essence forms a ‘window’ that contains 1000 tags from some point in the genome. If the reference point of a window begins from chromosome 1, in this instance the window of virtual tags 1 through 1000, then the number of tags that were actually sequenced from within this window is counted. Then the window is moved one virtual tag (now contains the region of chromosome 1 that has virtual tag 2 through tag 1001), and number of tags that were found is counted. This window can slide one virtual tag at a time, and the ratio of sequenced tags to virtual tags (always 1000) with the window will indicate what the tag density is.

Tag densities can be evaluated over moving windows to detect amplifications, deletions and other abnormalities. An aspect of this invention further comprises identifying a region of deletion or amplification comprising calculating the density of tags in a sliding window across the chromosome. An increase or a decrease in density is indicative of amplification or deletion, respectively.

Another aspect of the invention is a method of mapping a class IIB restriction enzyme tag sequence to its location in the genome comprising the above method steps, and further comprising sequencing the tag and comparing the sequence to markers in the genome. The presence of foreign DNA can be detected by analyzing those sequence tags which have no apparent matches to the human genome, and comparing the sequences to databases containing sequences derived from pathogens.

### EXAMPLES

### Example 1

### Construction of Karyotyping Libraries

Two experimental libraries were made using DNA purchased from ATCC (American Type Culture Collection, Manassas, VA). One library was made using DNA from a breast cancer cell line (primary ductal carcinoma, HCC38) and a reference library for karyotyping (and to emulate pathogen discovery) was made from the corresponding EBV (Epstein-Barr Virus) transformed blood cell line (HCC38 BL). 15 μg of DNA was digested using 60 units of BsaXI (New England Biolabs, Beverly, Mass.). After digestion, the reaction mix was phenol/chloroform washed and ethanol/ammoniumacetate precipitated (Maniatis ref). The digest was then run on an 8% polyacrylamide gel (200 volt/2.5 hours). The gel was stained using GelStar (Cambrex, East Rutherford, N.J.), and the band corresponding to the BsaXI tags was excised. The tags were purified using the crush-'n-soak method (Maniatis ref), and dissolved in 39.5 μl dH₂O and 5 μl Eco Pol Buffer (New England Biolabs, Beverly, Mass.). Blunting was done by adding 5 units large fragment DNA polymerase I (‘Klenow’, New England Biolabs, Beverly, Mass.) in the presence of 33 μM of each dNTP for 15 minutes at 25° C. in a total volume of 50 μl. After blunting, tags were washed/precipitated (see above) and dissolved in 7 μl dH₂O with 1 μl of 10× T4 DNA ligase buffer (New England Biolabs, Beverly, Mass.). The primary ligation was done by adding 200 ng of vector, 1 ul of high concentration T4 DNA ligase (New England Biolabs, Beverly, Mass.) and incubating overnight at 16° C. The vector used was an EcoRV cleaved, dephosphorylated PUC19 plasmid (Invitrogen) that had been modified to contain two PstI sites immediately flanking the EcoRV site. Ligations were washed/precipitated, and electrocompetent E. cloni 10G Elite cells (Lucigen, Middleton, Wis.) were transformed according to manufacturers' recommendations. After electroporation followed by 1 hour incubation in TB medium, the transformations were transferred to 250 ml TB medium containing 75 μg/ml ampicillin. Cells were grown to OD600 reached approximately 1.6 (about 13 hours) and plasmids were purified using a QIAfilter Plasmid Maxi kit (Qiagen, Valancia, Calif.). 200 μg of plasmids were digested using 1000 units of PstI (New England Biolabs, Beverly, Mass.). Digests were washed/precipitated and run on an 8% polyacrylamide gel (200 volt, 20 minutes—sufficient to separate released inserts from opened vector). Released tags were purified as described above and dissolved in 8 μl dH₂O and 1 μl 10× T4 DNA ligase buffer. 1 μl of high concentration T4 DNA ligase was added and the concatenation reaction was incubated for 1 hour at 16° C. Concatenates were loaded directly on a 13 cm agarose gel (1.5%) containing GelStar and run for 1.5 hours (125 volt). Concatenation products between 1200 bp and approximately 3000 bp were gel-purified using MinElute gel extraction kit (Qiagen, Valencia, Calif.). Concatenates were cloned in a PstI-cleaved p-ZeRO-1 vector, and the secondary transformations were done using E. cloni 10G Elite cells as described above. Concatenates were sequenced by SeqWright (Houston, Tex.).

### Example 2

### Library from Whole Genome Amplified DNA

Genomic human DNA was purchased from Clonetch (San Jose, Calif.) and 100 ng was whole genome amplified (WGA) using the REPLI-g kit (Molecular Staging, New Haven, Conn.) according to manufacturers' recommendations. After WGA, 80 μg of DNA was phenol/chloroform washed and ethanol/ammoniumacetate precipitated. The DNA was digested using 500 units of BsaXI, and tags were purified as described above. Purified tags were dissolved in 8 μl dH2O and 1 μl of 10× T4 DNA ligase buffer. Concatenates were made directly from the BsaXI tags by incubating them overnight at 16° C. with 1 ul of high concentration T4 DNA ligase. Concatenates were then run directly on a 1.5% agarose gel, and large concatenates were gel-purified as described above. Concatenates were blunted using 5 units of Klenow (as described for BsaXI tags above) and cloned in 200 ng of EcoRV-cleaved p-ZeRO-1 vector. The transformation was done using E. cloni 10G Elite cells and concatenates were sequenced by Agencourt (Boston, Mass.).

### Example 3

### Hybridization of Tags to Microarrays

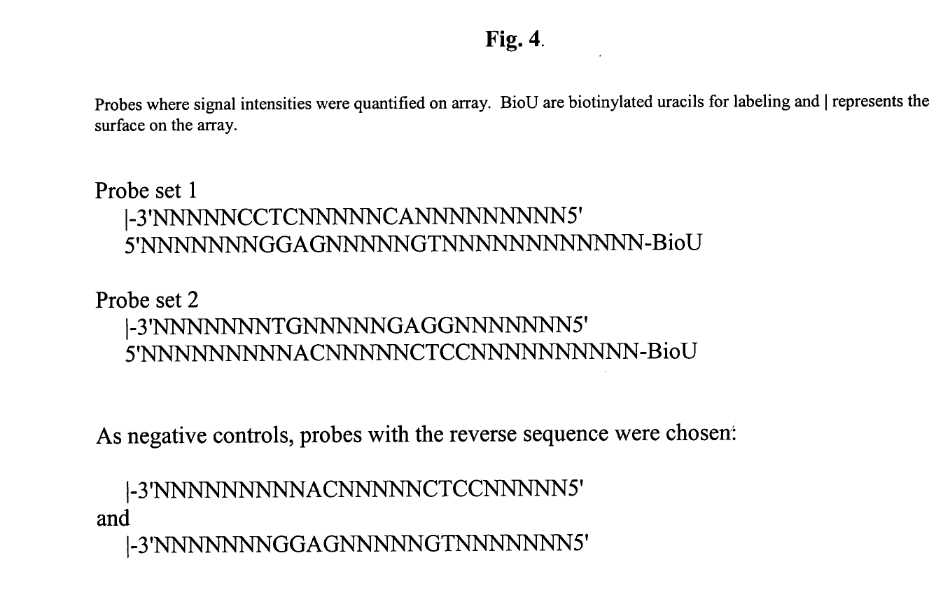

As a pilot experiment, we generated tags using 40 μg of normal human DNA and BsaXI as the Type IIB restriction enzyme (150-200 ng tags). Tags were polyacrylamide purified and 3′ end-labeled using terminal transferase and biotinylated uracil (standard protocol for labeling of DNA fragments for Affymetrix 10K SNP array). Standard Affymetrix hybridization and analysis was done and probes on the array corresponding to BsaXI sites were investigated (‘BsaXI probe sites’). Probes where chosen that hybridized in an optimal way to BsaXI tags (FIG. 4). Positive control hybridizations were also done using materials and protocols provide by Affymetrix. For all the matching probes, there is also a corresponding mismatch probe on the 10K SNP array. This probe has a mismatch in the central base and can be used to assess the specificity of the hybridization. We also chose to check signal intensity from probes with similar base composition that should not hybridize to our BsaXI tags (reverse sequence probes).

The results (FIG. 5) show that the BsaXI tags hybridize very specifically and effectively to the chip, with absolute signal intensities and specificity of hybridization similar to the Affymetrix positive control. Signal from our negative sequence probes was very low (76-77), and our Probe set 2 actually gave a higher ratio of signal/noise (match probe/mismatch probe) than Affymetrix control DNA.

### Example 4

### Karyotyping

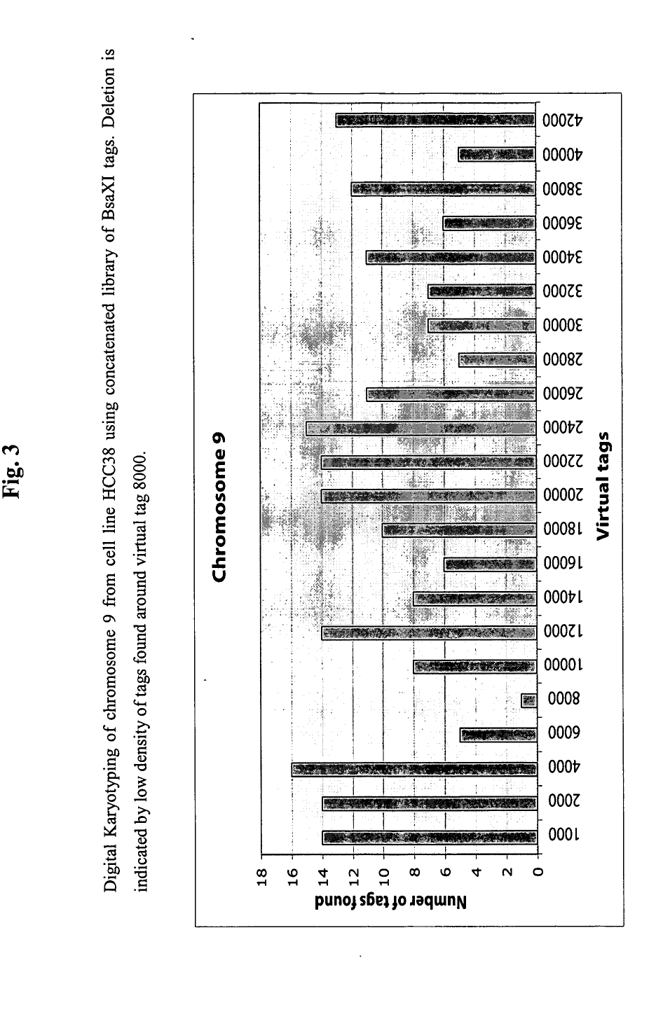

As described above, tags can be mapped to their locations in the genome, and regions where there is a lower or higher tag-count can be identified. These regions may represent loci that are amplified or deleted (homozygous or heterozygous deletions). As a pilot experiment, we generated 6500 unique tags from a breast cancer cell-line (HCC38, from ATCC) with known regions of deletion and amplification (Zhao et al. submitted). Chromosome 9 has an 11 megabase deletion, and by calculating density of tags found (based on the number of cut-sites—‘virtual tags’) in a sliding window across the chromosome, we were also able to identify this deletion (FIG. 3).

### Example 5

### Pathogen Discovery

Tags that do not match the published human genome or transcriptome may represent infectious agents present in the specimen the DNA/RNA was extracted from. This approach can be used to identify novel pathogens in disease that appear infectious but where the pathogen has not yet been characterized.

To test the feasibility of this approach to pathogen discovery, a concatenated BsaXI tag library was made using DNA from cell line HCC38 BL (ATCC). This cell line has been transformed using Epstein-Barr virus (EBV, Human herpesvirus 4). Using sequence information from 1347 Type IIB restriction enzyme tags (96 sequencing reactions), six different tags with perfect matches to the published wildtype EBV genome, were identified.

### Example 6

### Computational Subtraction of BsaXI Tags

For computational subtraction, a RECORD library was made uisng DNA from an EBV (Epstein-Barr virus) containing cell line. To test the accuracy of the subtraction, a control dataset was also made by computationally extracting 9,989 tags from 19,542 complete known microbe genomes. MegaBLAST was used for the initial subtraction. Tags were sequentially compared to phase 0, 1, 2 and 3 and build 34 of the human genome as well as the mitochondrial genome. High scoring tags were removed, and a second round of subtraction was done using BLAST. The BLAST database comprised phase0, 1, 2 and 3, build 34, and the mitochondrial genome. Tags were ranked according to bit scores, and high scoring tags were sequentially subtracted, as shown in the table below.

| | Database | | | | | |
|---|---|---|---|---|---|---|
| | (above) | | % microbe | | % non-EBV | |
| | Bit score | # microbe tags | tags | Non-EBV | tags | |
| | (below) | from databases | remaining | tags: | remaining | EBV tags: |
| Mega | | 19,542 | 100 | 19,890 | 100 | 44 |
| BLAST* | phase0 | 19,442 | 99.49 | 14,642 | 73.51 | 44 |
| | phase1 | 19,048 | 97.47 | 7,202 | 36.27 | 44 |
| | phase2 | 19,034 | 97.40 | 6,940 | 34.96 | 44 |
| | phase3 | 18,621 | 95.29 | 294 | 1.68 | 41 |
| | build 34 | 18,613 | 95.25 | 289 | 1.65 | 41 |
| | Mitochondrial | 18,613 | 95.74 | 282 | 1.62 | 41 |
| BLAST** | <42 | 18,613 | 95.25 | 282 | 1.41 | 41 |
| | <40 | 17,886 | 91.53 | 280 | 1.40 | 39 |
| | <38 | 13,059 | 66.83 | 116 | 0.58 | 31 |
| | <36 | 7,090 | 36.28 | 14 | 0.07 | 17 |
| | <34 | 2,891 | 14.79 | 3 | 0.02 | 11 |
| | <32 | 1,505 | 7.70 | 0 | 0 | 5 |
| | <30 | 119 | 0.61 | | | 0 |
| | <28 | 0 | 0 | | | |

*MegaBLAST analyses (word size 12, score for match 1 and penalty for mismatch −2) were done, and tags were sequentially compared to the databases. Tags with bit scores above 40 were removed.
**BLAST analyses (word size 7, E value 1,000, cost to open gap 5, cost to extend gap 2 and −3 as penalty for nucleotide mismatch) were performed, and tags with bit scores above specific values were subtracted.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. A method of making a nucleic acid RECORD library comprising contcatemers of type IIB restriction enzyme tags, comprising:
a) generating type IIB restriction enzyme tags from a biological sample,
b) forming concatemers of the type IIB restriction enzyme tags generated in step a), and
c) cloning said concatemers,
thereby forming a library comprising concatemers of type IIB restriction enzyme tags.

2. The method of claim 1, wherein step (a) comprises:
(i) digesting DNA from a biological sample with a type IIB restriction enzyme,
(ii) making the ends of the type IIB restriction enzyme tags produced in step (i) blunt, and
(iii) converting the blunt ends of the type IIB restriction enzyme tags produced in step (ii) to ends recognized by a punctuating restriction enzyme.

3. The method of claim 1 wherein step (b) comprises ligating the type IIB restriction enzyme tags to each other, thereby producing concatemers of the tags.

4. The method of claim 3, wherein each of the concatemers has a length from about 1000 to about 2000 base pairs.

5. The method of claim 1 wherein the DNA is genomic DNA or cDNA.

6. The method of claim 5, wherein the DNA is cDNA.

7. The method of claim 1, wherein the DNA was generated from a vertebrate.

8. The method of claim 7, wherein said veterbrate is a mammal.

9. The method of claim 8, wherein said mammal is a human.

10. The method of claim 1, wherein the Class IIB restriction enzyme is selected from the group consisting of AloI, PpiI, PsrI, BaeI, BplI, FalI, BcgI, Bsp24I, BsaXI, CjeI, CjePI, HaeIV and Hin4I.

11. The method of claim 10, wherein the Class IIB restriction enzyme is BsaXI.

12. A RECORD nucleic acid library comprising a plurality of concatemers comprising Type IIB restriction enzyme tags, wherein each Type IIB restriction enzyme tag comprises a Type IIB restriction enzyme recognition site flanked by cleavage sites recognized by a Type IIB restriction enzyme.

13. The RECORD library of claim 12, wherein said concatenated Type IIB restriction enzyme tags are linked by a recognition site for a punctuating restriction enzyme.

14. The RECORD library of claim 12, wherein the length of each said tag is greater than 20 base pairs.

15. The RECORD library of claim 12, wherein each of the concatemer consists of a length from about 1000 to about 2000 base pairs.

16. The RECORD library of claim 12, wherein the library comprises more than 1,000 concatemers.

17. The RECORD library of claim 12, wherein the Type IIB restriction enzyme tags were generated from a vertebrate.

18. The RECORD library of claim 17, wherein said veterbrate is a mammal.

19. The RECORD library of claim 18, wherein said mammal is a human.

20. The RECORD library of claim 12, wherein said Type IIB restriction enzyme tags are generated from cDNA.

21. The RECORD library of claim 12, wherein said Type IIB restriction enzyme tags are generated from genomic DNA.

22. The RECORD library of claim 12, wherein the Class IIB restriction enzyme is selected from the group consisting of AloI, PpiI, PsrI, BaeI, BplI, FalI, BcgI, Bsp24I, BsaXI, CjeI, CjePI, HaeIV and Hin4I.

23. The RECORD library of claim 22, wherein the Class IIB restriction enzyme is BsaXI.

24. A kit for generating the RECORD library of claim 12 from a DNA sample of interest, comprising a detailed protocol for RECORD library construction, two cloning vectors, a shuttle vector for monomer cloning, a destination vector for concatemer cloning, Type IIB restriction endonuclease, T4 DNAse ligase or equivalent, DNA polymerase Klenow fragment, or equivalent, restriction enzymes that recognize the punctuating restriction sequence, and buffers and nucleotides.

25. A method of identifying a pathogen in a biological sample comprising;
(i) sequencing the concatenated tags in the RECORD library of claim 12, wherein said tags were generated from the biological sample,
(ii) identifying a sequence of a tag, wherein said sequence or the complement thereof, is not present in a corresponding uninfected or non-diseased sample,
(iii) identifying a pathogen that comprises the sequence of part ii, by comparison to nucleic acid sequences from pathogenic organisms.

26. A method for identifying a pathogen in a biological sample, comprising;
i) preparing Type IIB restriction enzyme tags from said biological sample,
ii) denaturing said Type IIB restriction enzyme tags, thereby forming single stranded tags,
iii) hybridizing said single stranded tags to a solid support containing a microarray of nucleic acid oligomeric probes, wherein said probes represent nucleic acid sequences derived from one or more pathogens, and
ii) detecting hybridization of said tags to the nucleic acid probes on said solid support, wherein the hybridization of one or more tags to one or more probes represents the presence of the said one or more pathogens in said sample.

27. The method of claim 26, wherein the step of preparing said Type IIB restriction enzyme tags of step i) comprises digesting concatemers of said tags with a punctuating enzyme which recognizes the recognition sequence used to link the concatemers, and isolating the digested tags.

28. The method of claim 27, wherein said concatenates are comprised by the RECORD library of claim 18.

29. A method of identifying one or more regions of deletion, amplification or chromosomal alteration in the genome of an animal, comprising;
(i) sequencing the tags in the library of claim 12, wherein said RECORD library is generated from a biological sample derived from said animal,
(ii) matching the sequence data with precise chromosomal locations,
(iii)calculating the density of tags across the chromosome, wherein an increase or a decrease in density is indicative of an amplification or deletion, respectively.

30. A method for the detection of expression of one or more transcripts of interest in a biological sample, comprising;
(i) sequencing at least one concatemer of Type IIB restriction enzyme tags comprised by the library of claim 20, wherein said library is produced from nucleic acid generated from said sample,
(ii) comparing the sequence of said tags with the sequence of the transcript of interest, wherein if the sequence of at least one of the tags corresponds to the sequence of said transcript, then the expression of said transcript in said sample has been detected.

31. A method for detecting the expression of one or more genes in a biological sample, comprising;
ii) preparing Type IIB restriction enzyme tags from said biological sample,
ii) denaturing said Type IIB restriction enzyme tags, thereby forming single stranded tags,
iii) hybridizing said single stranded tags to a solid support containing a microarray of nucleic acid oligomeric probes, and
ii) detecting hybridization of said tags to the nucleic acid probes on said solid support, wherein the hybridization of one or more tags to one or more probes represents the expression of the said one or more genes in said sample.

32. The method of claim 31, wherein the step of preparing said Type IIB restriction enzyme tags of step i) comprises digesting concatemers of said tags with a punctuating enzyme which recognizes the recognition sequence used to link the concatemers, wherein said concatemers are produced from cDNA generated from RNA of said sample, and isolating the digested tags.

33. The method of claim 32, wherein said concatenates are comprised by the RECORD library of claim 13, and wherein said library is produced from cDNA generated from said sample,

34. The method of claim 31, wherein said probes consist of a length identical to the length of said tags.

35. The method of claim 31, wherein said probes consist of a length of between 27 and 32 base pairs.

36. The method of claim 31, wherein said tags are labeled.

37. The method of claim 31, wherein said probes are selected from the group consisting of ESTs, RNAs, CDNAs and type IIB restriction enzyme tags.

38. The method of claim 31, wherein said probes target only tags which are unique in the genome.

39. A method to generate a subtractive library of type IIB restriction enzyme tags generated from an experimental sample SORT, comprising;
(i) separately generating type IIB restriction enzyme tags from an experimental sample and from a control sample,
(ii) attaching the labeled tags to a solid surface,
(iii) hybridizing the experimentally generated tags to the attached labeled tags, and p1 (iv) purifying the unbound tags,
thereby generating a subtractive library type IIB restriction enzyme tags.

40. The method according to claim 39, wherein the solid surface is selected from the group consisting of a chip, a nylon membrane, derivatized glass, or silicon.

41. A chip that contains an array of nucleic acid probes comprising type IIB restriction enzyme tags.

42. A kit comprising the chip of claim 41.

43. A method of mapping one or more nucleic acid molecules in a biological sample to its respective location in the genome of an animal, comprising;
(i) sequencing the tags in the library of claim 12, wherein said library is generated from a biological sample derived from said animal,
(ii) comparing the sequence of a specific tag to markers in the genome of the animal from which the tag was derived,
thereby determining the location of a class IIB tag in the genome.

44. A method of identifying a pathogen in a biological sample comprising:
a) generating a RECORD library comprising concatemers of type IIB restriction enzyme tags, wherein said tags are generated from the biological sample suspected of comprising the pathogen,
b) sequencing the concatenated tags in the library,
c) identifying a tag whose sequence or complement thereof, is not present in a corresponding uninfected or non-diseased sample,
d) identifying a candidate pathogen by its absence in the reference sample or genome.

45. The method of claim 44, wherein the identification of a tag whose sequence or complement thereof, is not present in a corresponding uninfected or non-diseased sample, comprises computational subtraction against the human genome.

46. The method of claim 44, wherein the identification of the pathogen is made by comparison to nucleic acid sequences from microbial organisms.

47. The method of claim 44, wherein the biologic sample is selected from the group consisting of genomic double stranded DNA, single stranded genomic DNA, messenger RNA or total RNA.

48. The method of claim 44, wherein the pathogen is an infectious disease organism.

49. The method of claim 44, wherein said pathogen is associated with a pathogenic condition selected from the group consisting of an inflammatory disease, an autoimmune disease, and a cell proliferative disease.

50. The method of claim 44, wherein said wherein the identification of a tag whose sequence or complement thereof, is not present in a corresponding uninfected or non-diseased sample, comprises using a computer system.

51. The method of claim 50, wherein said computer system comprises obtaining sequence information from at least one host organism having a pathogenic condition, b) identifying sequences from said at least one host organism which are not found in a plurality of host organisms not having said pathogenic condition; C) comparing said sequences identified in step (b) with a plurality of sequences in a database of host genomic sequences; and d) eliminating identified sequences which match said host genomic sequences, wherein any remaining sequences are identified as candidate pathogen sequences.

52. The method according to claim 51, wherein said identified sequences are compared simultaneously with sequences in said database of host genomic sequence.

53. The method according to claim 44, wherein said biological sample is derived from a host organism.

54. The method according to claim 53, wherein said host organism is an animal.

55. The method according to claim 54, wherein said animal is a mammal.

56. The method according to claim 55, wherein said mammal is a human.

57. The method according to claim 54, wherein said animal is an insect, bird, or a fish.

58. The method according to claim 53, wherein said host organism is a microorganism, a fungus, or a plant.

59. A method of generating a subtractive library comprising Type IIB Restriction enzyme tags, (SORT) comprising,
A) generating a collection of Type IIB restriction tags from a sample, wherein the sample represents a control nucleic acid population,
B) immobilizing the collection of tags generated in step A to a solid support,
C) generating an independent collection of Type IIB restriction tags from a second sample, wherein said second sample represents an experimental nucleic acid population,
D) hybridizing said second set of tags to the immobilized tags of step b),
E) Collecting tags from said second set of tags which do not hybridize to the immobilized tags of step b),
wherein said collected tags from step E) represent said subtractive library comprising Type IIB Restriction enzyme tags.

60. The method of claim 59, wherein said solid support is selected from the group consisting of a bead, a column, a filter, a membrane or an array.

61. The method according to claim 59, wherein said control nucleic acid population is normal human genomic DNA.

62. The method according to claim 59, wherein said experimental nucleic acid population is generated from a candidate infected tissue or a cancer specimen.

63. The method according to claim 59, wherein steps c-e are repeated two or more times.

64. The method according to claim 59, wherein the Type IIB restriction tags generated from said second sample in step c of claim 59, are modified to contain linkers, wherein said linkers contain sequences allowing the amplification, cloning, concatenation into RECORD libraries and sequencing.

65. A method of discovering a pathogen comprising generating a subtractive library according to the method of claim 16, wherein said second set of tags are generated from a biological sample derived from a host suspected of comprising the pathogen, wherein the first set of tags is generated from a biological sample derived from a corresponding host that is uninfected or non-diseased, and wherein the generated subtractive library represents one or more sequences derived from a pathogen, and wherein sequencing said one or more sequences leads to the identification and discovery of said pathogen.

66. A method of discovering a genomic alteration, comprising generating a subtractive library according to the method of claim 16, wherein said second set of tags are generated from a biological sample derived from a cell suspected of containing a genomic alteration, wherein the first set of tags is generated from a biological sample derived from a corresponding cell not containing said genomic alteration, and wherein the generated subtractive library represents one or more sequences which contains said alteration, and wherein sequencing said one or more sequences leads to the identification and discovery of said genomic variation.

67. The kit according to claim 24, further comprising a detailed protocol for making doublestranded DNA from RNA, reverse transcriptase, RNASe H, DNA polymerase, nucleotides and buffers.

68. The kit according to claim 24 or 67, further comprising reagents to enrich for tags of interest SORT, before making the RECORD library, wherein said kit further comprises instructions for SORT, type IIB restriction enzymes, ligase, linkers, a solid support, means to attach tags derived from the control sample to a solid support.

69. The method of claim 1, wherein said type IIB restriction enzyme tags were generated from the subtractive library of claim 39.

70. The subtractive library of claim 39.


SEQUENCE LISTING (3 sequences)

SEQ ID NO: 1 - DNA, 30 bp, Artificial
  one strand of DNA tag released by Restriction Enzyme BsaXI
nnnnnnnnnacnnnnnctccnnnnnnnnnn

SEQ ID NO: 2 - DNA, 30 bp, artificial sequence
  one strand of tag released by restriction enzyme BsaXI
nnnnnnnggagnnnnngtnnnnnnnnnnnn

SEQ ID NO: 3 - DNA, 11 bp, artificial sequence
  the recognition site of CjeI
ccannnnnngt